(19) United States
(12) Patent Application Publication
(10) Pub. No.: US 2013/0341207 A1
(43) Pub. Date: Dec. 26, 2013

(54) ANALYTICAL TEST STRIP WITH CAPILLARY SAMPLE-RECEIVING CHAMBERS SEPARATED BY STOP JUNCTIONS

(71) Applicants: Neil WHITEHEAD, Inverness (GB); Scott SLOSS, Inverness (GB); Antony SMITH, Inverness (GB); Lynsey WHYTE, Inverness (GB); David MCCOLL, Inverness (GB); David HOWIE, Inverness (GB); Ramsay DARLING, Inverness (GB); James MCLAREN, Inverness (GB)

(72) Inventors: Neil WHITEHEAD, Inverness (GB); Scott SLOSS, Inverness (GB); Antony SMITH, Inverness (GB); Lynsey WHYTE, Inverness (GB); David MCCOLL, Inverness (GB); David HOWIE, Inverness (GB); Ramsay DARLING, Inverness (GB); James MCLAREN, Inverness (GB)

(73) Assignee: LifeScan Scotland Limited

(21) Appl. No.: 13/529,879

(22) Filed: Jun. 21, 2012

(51) Int. Cl. G01N 27/26; G01N 33/66

(52) U.S. Cl. 205/782; 204/403.03; 205/792

(57) ABSTRACT

An analytical test strip for the determination of an analyte (such as glucose) in a bodily fluid sample (e.g., a whole blood sample) includes a first and second capillary sample-receiving chambers and first and second stop junctions that are disposed between the first and second capillary sample-receiving chambers. The first stop junction defines a discontinuity boundary of the first capillary sample-receiving chamber and the second stop junction defines a discontinuity boundary of the second capillary sample-receiving chamber. In addition, the first stop junction and the second stop junction are disposed such that bodily fluid sample flow between the first capillary sample-receiving chamber and the second capillary sample-receiving chamber during use of the analytical test strip is prevented.

### BACKGROUND OF THE INVENTION

1. Field of the Invention

The present invention relates, in general, to medical devices and, in particular, to analytical test strips and related methods.

2. Description of Related Art

The determination (e.g., detection and/or concentration measurement) of an analyte in a fluid sample and/or the determination of a characteristic of a fluid sample (such as haematocrit) are of particular interest in the medical field. For example, it can be desirable to determine glucose, ketone bodies, cholesterol, lipoproteins, triglycerides, acetaminophen and/or HbA1c concentrations in a sample of a bodily fluid such as urine, blood, plasma or interstitial fluid. Such determinations can be achieved using analytical test strips, based on, for example, visual, photometric or electrochemical techniques. Conventional electrochemical-based analytical test strips are described in, for example, U.S. Pat. Nos. 5,708,247, and 6,284,125, each of which is hereby incorporated in full by reference.

### BRIEF DESCRIPTION OF THE DRAWINGS

The accompanying drawings, which are incorporated herein and constitute part of this specification, illustrate presently preferred embodiments of the invention, and, together with the general description given above and the detailed description given below, serve to explain features of the invention, in which:

FIG. 1 is a simplified exploded view of an electrochemical-based analytical test strip according to an embodiment of the present invention;

FIG. 2 is a sequence of simplified top views of the various layers of the electrochemical-based analytical test strip of FIG. 1;

FIG. 3 is a simplified top view representation of the substrate layer and spacer layer of the electrochemical-based analytical test strip of FIG. 1 that includes dashed lines to delineate the first stop junction and the second stop junction of the electrochemical-based analytical test strip;

FIG. 4 is a simplified side view of a portion of the electrochemical-based analytical test strip of FIG. 1 that, for clarity, omits the reagent layer, patterned insulation layer and patterned conductor layer thereof;

FIG. 5 is a simplified top view of the electrochemical-based analytical test strip of FIG. 1 depicting various components thereof;

FIG. 6 is a simplified side view of a portion of an electrochemical-based analytical test according to another embodiment of the present invention that, for clarity, omits the reagent layer, patterned insulation layer and patterned conductor layer thereof;

FIG. 7 is a simplified side view of a portion of an electrochemical-based analytical test according to yet another embodiment of the present invention, for clarity, omits the reagent layer, patterned insulation layer and patterned conductor layer thereof;

FIG. 8 is a simplified side view of a portion of an electrochemical-based analytical test according to still another embodiment of the present invention, for clarity, omits the reagent layer, patterned insulation layer and patterned conductor layer thereof; and

FIG. 9 is a flow diagram depicting stages in a method for determining an analyte in a bodily fluid sample according to an embodiment of the present invention.

### DETAILED DESCRIPTION OF ILLUSTRATIVE EMBODIMENTS

The following detailed description should be read with reference to the drawings, in which like elements in different drawings are identically numbered. The drawings, which are not necessarily to scale, depict exemplary embodiments for the purpose of explanation only and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “about” or “approximately” for any numerical values or ranges indicate a suitable dimensional tolerance that allows the part or collection of components to function for its intended purpose as described herein.

In general, analytical test strips (e.g., electrochemical-based analytical test strips) for the determination of an analyte (such as glucose) in a bodily fluid sample (for example, whole blood) according to embodiments of the present invention include first and second capillary sample-receiving chambers and first and second stop junctions that are disposed between the first and second capillary sample-receiving chambers. The first stop junction defines a discontinuity boundary of the first capillary sample-receiving chamber and the second stop junction defines a discontinuity boundary of the second capillary sample-receiving chamber. In addition, the first stop junction and the second stop junction are disposed such that bodily fluid sample flow between the first capillary sample-receiving chamber and the second capillary sample-receiving chamber is prevented during use of the analytical test strip.

When fluid flows through a capillary channel or chamber, a discontinuity in surface tension can cause a back pressure that prevents the fluid from proceeding through the discontinuity. Such a discontinuity is referred to as a “stop junction” and can be caused by, for example, an abrupt change in channel cross-section (i.e., a change in a channel or chamber dimension) and/or a change in the hydrophilic and/or hydrophobic nature of the surfaces defining the channel or chamber. Stop junctions based on changes in channel cross-section are described, for example, in U.S. Pat. Nos. 6,488,827, 6,521,182 and 7,022,286, each of which is hereby incorporated in full by reference.

Analytical test strips according to embodiments of the present invention are beneficial in that, for example, the stop junction(s) serves to maintain the fluidic integrity of the first and second capillary sample-receiving chambers while also being relatively small and easily manufactured. Such fluidic integrity beneficially prevents mixing of reagents and reaction byproducts between the first and second capillary sample-receiving chambers that can lead to inaccuracies in analyte or bodily fluid sample characteristic determination. Moreover, since the stop junctions are relatively small, sample application openings for the first and second capillary sample application chambers can be juxtaposed close to one another (for example, separated by a distance of approximately 250 microns that can be operatively bridged by a whole blood sample of approximately 1 micro-liter) such that the single application of a bodily fluid sample bridges both sample application openings and fills both the first and the second capillary sample-receiving chambers.

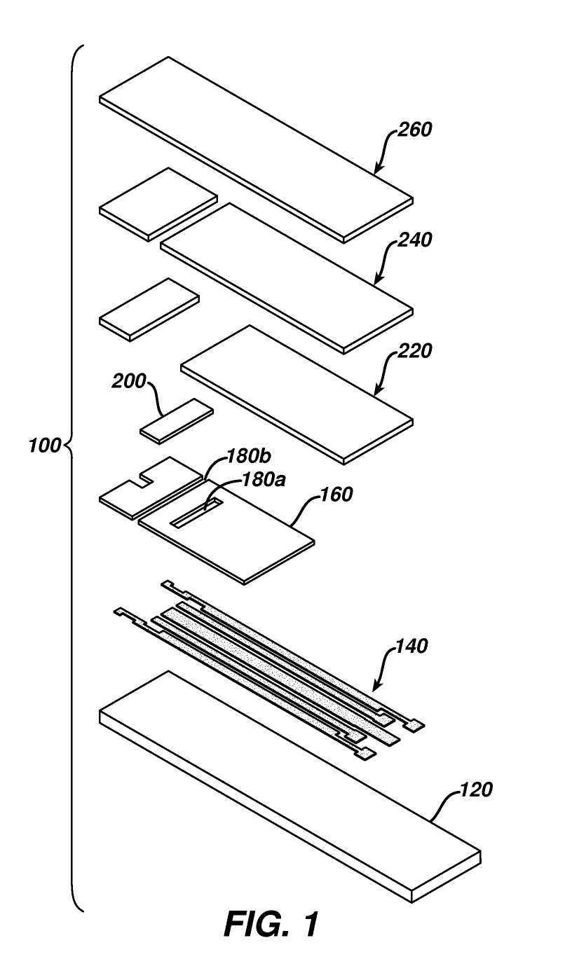

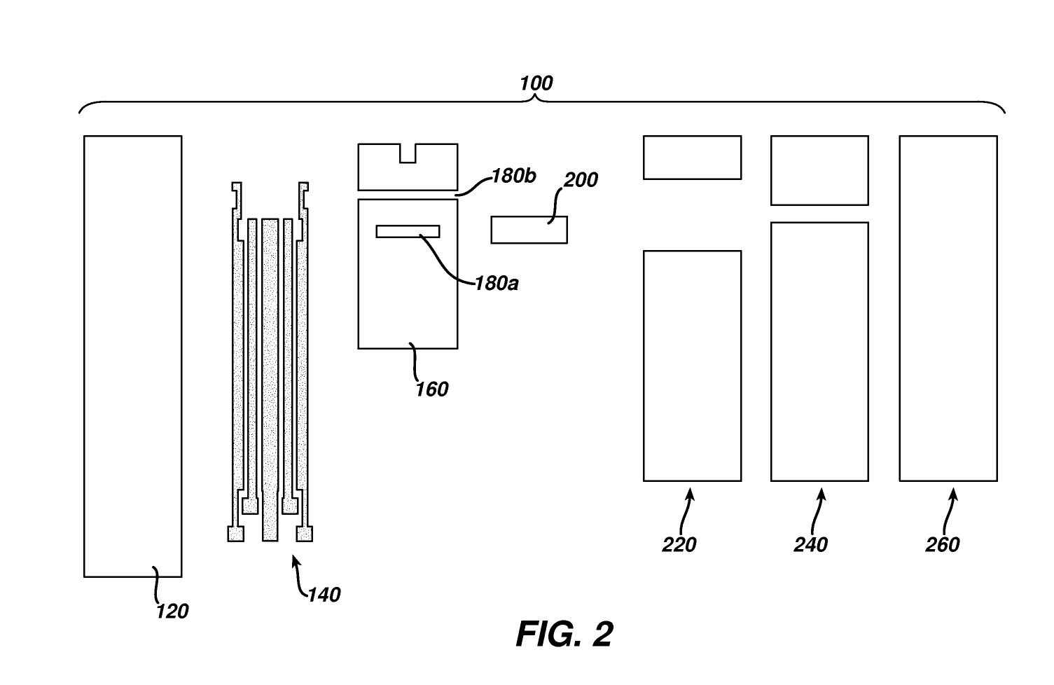

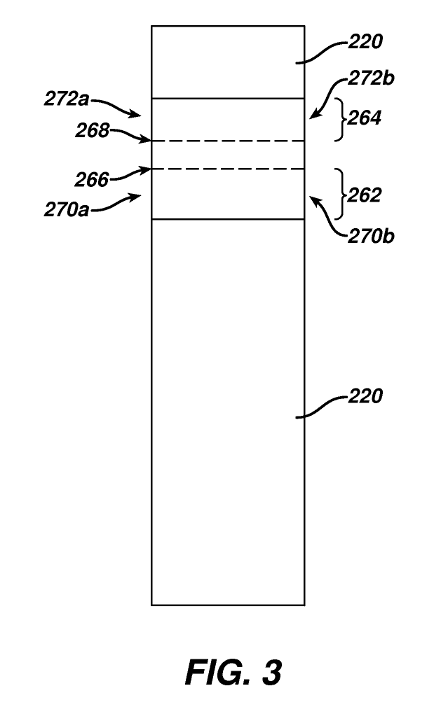

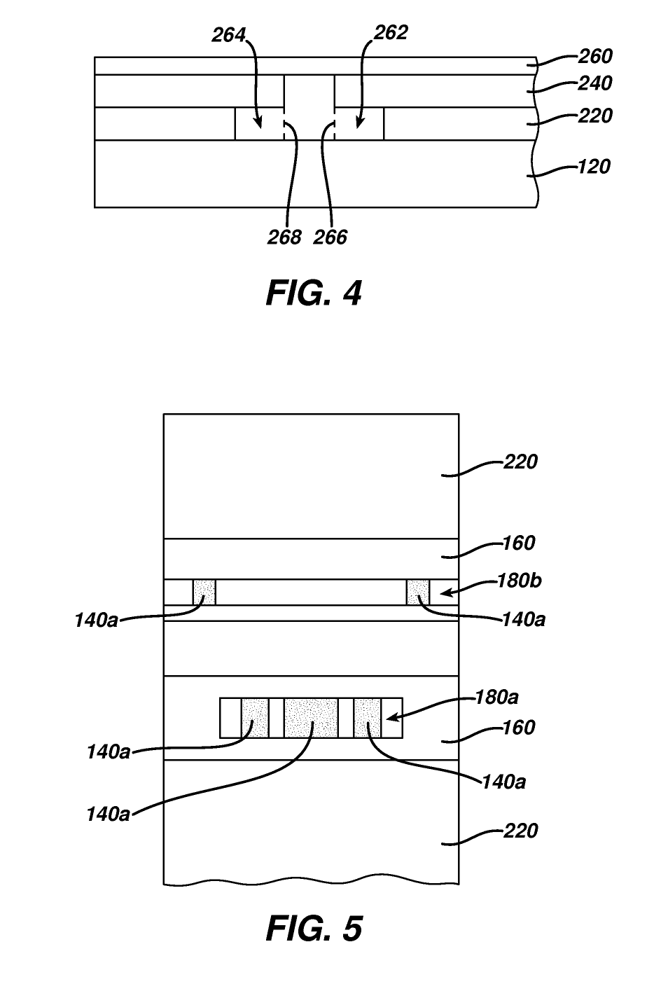

FIG. 1 is a simplified exploded view of an electrochemical-based analytical test strip 100 according to an embodiment of the present invention. FIG. 2 is a sequence of simplified top views of the various layers of electrochemical-based analytical test strip 100. FIG. 3 is a simplified top view representation of the substrate layer and spacer layer of electrochemical-based analytical test strip 100 that includes dashed lines to delineate the first stop junction and the second stop junction. FIG. 4 is a simplified side view of a portion of electrochemical-based analytical test strip 100 that, for clarity, omits the reagent layer, patterned insulation layer and patterned conductor layer thereof. FIG. 5 is a simplified top view of electrochemical-based analytical test strip 100 depicting various components, including the electrodes, thereof.

Referring to FIGS. 1-5, electrochemical-based analytical test strip 100 for the determination of an analyte (such as glucose) in a bodily fluid sample (for example, a whole blood sample) includes an electrically-insulating substrate layer 120, a patterned conductor layer 140, a patterned insulation layer 160 with electrode exposure windows 180a and 180b therein, an enzymatic reagent layer 200, a patterned spacer layer 220, a hydrophilic layer 240, and a top layer 260.

The disposition and alignment of electrically-insulating substrate layer 120, patterned conductor layer 140 (which a variety of electrodes 140a, see FIG. 5 in particular), patterned insulation layer 160, enzymatic reagent layer 200, patterned spacer layer 220, hydrophilic layer 240 and top layer 260 of electrochemical-based analytical test strip 100 are such that a first capillary sample-receiving chamber 262 and a second capillary sample-receiving chamber 264 are defined.

Moreover, the disposition is also such that a first stop junction 266 (delineated by dashed lines in FIGS. 3 and 4) is formed and disposed between first capillary sample-receiving chamber 262 and the second capillary receiving chamber 264, with the first stop junction defining a discontinuity boundary of first capillary sample-receiving chamber 262. Furthermore, the disposition is such that a second stop junction 268 (delineated by dashed lines in FIGS. 3 and 4) is disposed between first capillary sample receiving chamber 262 and second capillary receiving chamber 264 and defining a discontinuity boundary of second capillary receiving chamber 264.

The first stop junction and the second stop junction are disposed such that bodily fluid sample flow between the first capillary sample-receiving chamber and the second capillary sample-receiving chamber during use of the analytical test strip is prevented. In the embodiment of FIGS. 1-5, such flow is prevented due to the abrupt change in a dimension (i.e., the vertical direction in the perspective of FIG. 4) of the first and second capillary sample-receiving chambers.

It should be noted that in the embodiments depicted in FIGS. 1-8. the first and second stop junctions are disposed essentially parallel to the primary flow direction of a bodily fluid that is filling the first and second sample receiving chambers. The first and second stop junctions, therefore, do not prevent bodily fluid from filling the first and second sample-receiving chambers but rather prevent bodily fluid that has entered either of the sample-receiving chambers from entering the other sample-receiving chamber.

In the perspective of FIG. 4, first and second capillary sample-receiving chambers 262 and 264 have a height of approximately 100 μm, a width in the range of approximately 1.45 mm to 1.65 mm, and a pitch of approximately 2.55 mm. The abrupt change in vertical dimension that creates the stop junctions is an additional height of approximately 100 μm.

Patterned conductor layer 104, including electrodes 140a, of analytical test strip 100 can be formed of any suitable material including, for example, gold, palladium, platinum, indium, titanium-palladium alloys and electrically conducting carbon-based materials including carbon inks. Referring in particular to FIG. 5, electrode exposure window 180a of patterned insulation layer 160 exposes three electrodes 140a (for example, a counter/reference electrode and first and second working electrodes) configured for the electrochemical determination of an analyte (glucose) in a bodily fluid sample (whole blood). Electrode exposure window 180b exposes two electrodes configured for the determination of haematocrit in whole blood. The determination of haematocrit using electrodes of an analytical test strip is described in, for example, U.S. Patent Application Nos. 61/581,100; 61/581,097; 61/581,089; 61/530,795 and 61/530,808, each of which is hereby incorporated in full by reference.

During use, a bodily fluid sample is applied to electrochemical-based analytical test strip 100 and fills both the first and second capillary sample-receiving chambers by capillary action and, thereby, operatively contacts the electrodes disposed in the first and second capillary sample-receiving chambers. Referring to FIG. 3 in particular, first capillary sample receiving chamber 262 has at least one sample application opening (namely two openings 270a and 270b) and second sample receiving chamber 264 has at least one sample application opening (namely, two sample openings 272a and 272b). Each of the first and second sample-receiving chambers are configured such that a sample can be applied and fill both of the chambers from either the left-hand side (using sample application openings 270a and 272a) of the analytical test strip or the right-hand-side (using sample application openings 270b and 272b). In either circumstance, the sample application opening of the first capillary sample-receiving chamber and the sample application opening of the second sample-receiving chamber are juxtaposed such that a single bodily fluid sample can be simultaneously applied thereto.

Electrically-insulating substrate layer 120 can be any suitable electrically-insulating substrate layer known to one skilled in the art including, for example, a nylon substrate, polycarbonate substrate, a polyimide substrate, a polyvinyl chloride substrate, a polyethylene substrate, a polypropylene substrate, a glycolated polyester (PETG) substrate, or a polyester substrate. The electrically-insulating substrate layer can have any suitable dimensions including, for example, a width dimension of about 5 mm, a length dimension of about 27 mm and a thickness dimension of about 0.35 mm.

Electrically-insulating substrate layer 120 provides structure to the strip for ease of handling and also serves as a base for the application (e.g., printing or deposition) of subsequent layers (e.g., a patterned conductor layer). It should be noted that patterned conductor layers employed in analytical test strips according to embodiments of the present invention can take any suitable shape and be formed of any suitable materials including, for example, metal materials and conductive carbon materials.

Patterned insulation layer 160 can be formed, for example, from a screen printable insulating ink. Such a screen printable insulating ink is commercially available from Ercon of Wareham, Mass. U.S.A. under the name “Insulayer.”

Patterned spacer layer 220 can be formed, for example, from a screen-printable pressure sensitive adhesive commercially available from Apollo Adhesives, Tamworth, Staffordshire, or other suitable materials such as, for example, polyester and polypropylene. The thickness of patterned spacer layer 220 can be, for example 75 um. In the embodiment of FIGS. 1 through 5, patterned spacer layer 220 defines an outer wall of the first and second capillary sample-receiving chamber 280.

Hydrophilic layer 240 can be, for example, a clear film with hydrophilic properties that promote wetting and filling of electrochemical-based analytical test strip 100 by a fluid sample (e.g., a whole blood sample). Such clear films are commercially available from, for example, 3M of Minneapolis, Minn. U.S.A. and Coveme (San Lazzaro di Savena, Italy). Hydrophilic layer 240 can be, for example, a polyester film coated with a surfactant that provides a hydrophilic contact angle <10 degrees. Hydrophilic layer 240 can also be a polypropylene film coated with a surfactant or other surface treatment, e.g., a MESA coating. Hydrophilic layer 240 can have a thickness, for example, of approximately 100 μm.

Enzymatic reagent layer 200 can include any suitable enzymatic reagents, with the selection of enzymatic reagents being dependent on the analyte to be determined. For example, if glucose is to be determined in a blood sample, enzymatic reagent layer 200 can include a glucose oxidase or glucose dehydrogenase along with other components necessary for functional operation. Enzymatic reagent layer 200 can include, for example, glucose oxidase, tri-sodium citrate, citric acid, polyvinyl alcohol, hydroxyl ethyl cellulose, potassium ferrocyanide, antifoam, cabosil, PVPVA, and water. Further details regarding enzymatic reagent layers, and electrochemical-based analytical test strips in general, are in U.S. Pat. Nos. 6,241,862 and 6,733,655, the contents of which are hereby fully incorporated by reference.

Top layer 260 can be formed of any suitable mater including, for example, polyester materials, polypropylene materials, and other plastic materials. Top layer 260 can have a thickness, for example of approximately 50 μm.

Electrochemical-based analytical test strip 100 can be manufactured, for example, by the sequential aligned formation of patterned conductor layer 140, patterned insulation layer 160, enzymatic reagent layer 200, patterned spacer layer 220, hydrophilic layer 240 and top layer 260 onto electrically-insulating substrate layer 120. Any suitable techniques known to one skilled in the art can be used to accomplish such sequential aligned formation, including, for example, screen printing, photolithography, photogravure, chemical vapour deposition and tape lamination techniques.

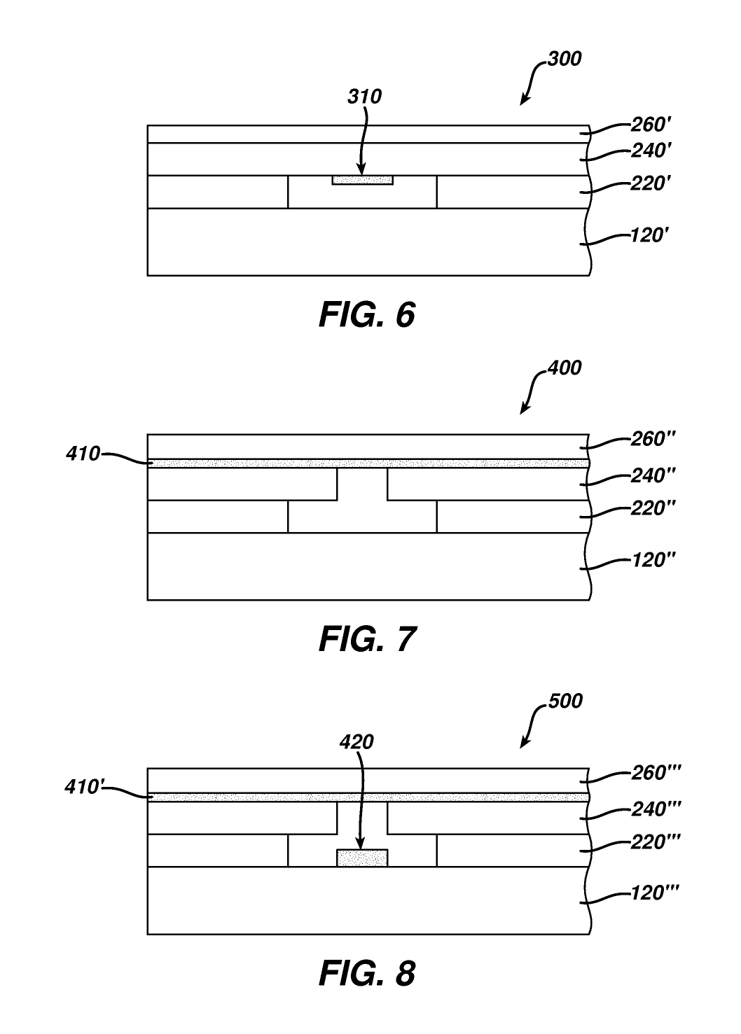

FIG. 6 is a simplified side view of a portion of an electrochemical-based analytical test 300 according to another embodiment of the present invention that, for clarity, omits the reagent layer, patterned insulation layer and patterned conductor layer thereof. Electrochemical-based analytical test strip 300 is similar to electrochemical-based analytical test strip 100 and a prime (′) has been added to component numbers that are similar. Electrochemical-based analytical test strip 300 differs, however, in that the first and second stop junctions are created by the presence of hydrophobic layer 310. Hydrophobic layer 310 can be formed, for example, from any suitable hydrophobic material such as a PTFE material, a carbon ink material or other suitable hydrophobic material with a contact angle of, for example, greater than 100 degrees.

FIG. 7 is a simplified side view of a portion of an electrochemical-based analytical test 400 according to yet another embodiment of the present invention, for clarity, omits the reagent layer, patterned insulation layer and patterned conductor layer thereof. Electrochemical-based analytical test strip 400 is similar to electrochemical-based analytical test strip 100 and a prime (′) has been added to component numbers that are similar. Electrochemical-based analytical test strip 400 differs, however, in that the first and second stop junctions are created by the additional presence of hydrophobic layer 410, as is evident by a comparison of FIGS. 4 and 7. In all other respected, electrochemical-based analytical test strip 400 is essentially identical to electrochemical-based analytical test strip 100.

Hydrophobic layer 410 can be formed, for example, from any suitable hydrophobic material such as a PTFE material, a carbon ink material, or other suitable hydrophobic material with a contact angle of, for example, greater than 100 degrees.

FIG. 8 is a simplified side view of a portion of an electrochemical-based analytical test according to still another embodiment of the present invention, for clarity, omits the reagent layer, patterned insulation layer and patterned conductor layer thereof. Electrochemical-based analytical test strip 500 is similar to electrochemical-based analytical test strip 100 and a prime (′) has, therefore, been added to component numbers that are similar. Electrochemical-based analytical test strip 500 differs, however, in that the first and second stop junctions are created by the additional presence of first hydrophobic layer 410′ and second hydrophobic layer 420, as is evident by a comparison of FIGS. 4 and 8. In all other critical respects, electrochemical-based analytical test strip 500 is essentially identical to electrochemical-based analytical test strip 100. First hydrophobic layer 410′ and second hydrophobic layer 420 can be formed, for example, from any suitable hydrophobic material such as a PTFE material or other suitable hydrophobic material with a contact angle of, for example, greater than 100 degrees.

In each of electrochemical-based analytical test strips 300, 400 and 500, their respective hydrophobic layers serve together create a surface tension-induced back-pressure that either fully defines the first and second stop junctions (see the embodiment of FIG. 6) or augments a surface tension-induced back-pressure created by a chamber height discontinuity (see the embodiments of FIGS. 7 and 8).

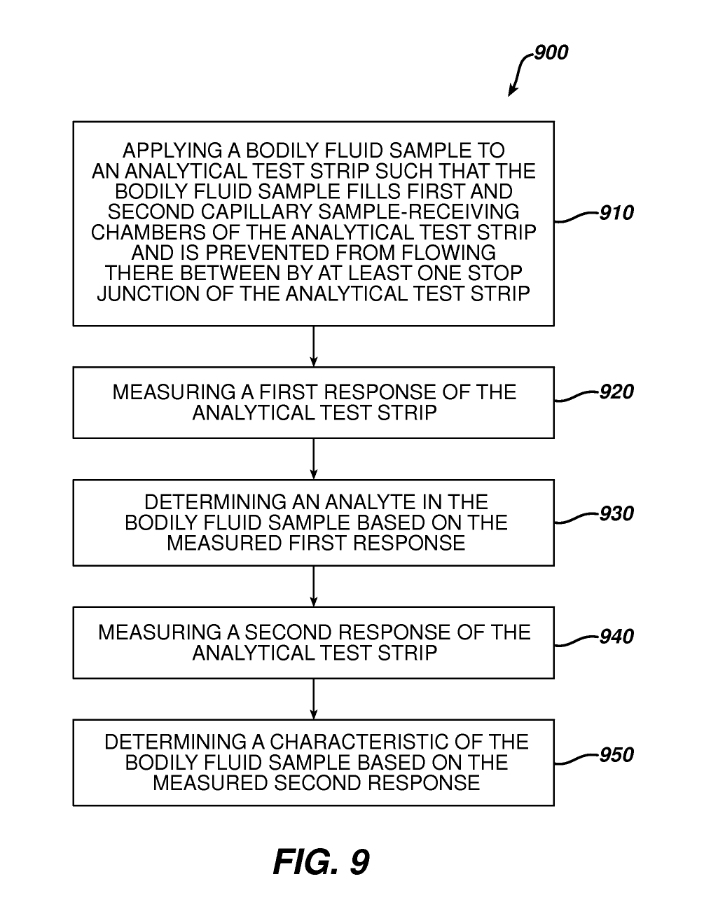

FIG. 9 is a flow diagram depicting stages in a method 900 for determining an analyte (such as glucose) in a bodily fluid sample (for example, a whole blood sample) and/or a characteristics of the bodily fluid sample (e.g., hematocrit) according to an embodiment of the present invention. Method 900 includes (see step 910 of FIG. 8) applying a bodily fluid sample to an analytical test strip such that the applied bodily fluid sample fills a first capillary sample-receiving chamber and a second capillary sample-receiving chamber of the analytical test strip and is prevented from flowing between the first capillary sample-receiving chamber and the second capillary sample-receiving chamber by at least one stop junction of either of the first capillary sample-receiving chamber and the second capillary sample-receiving chamber.

Method 900 also includes measuring a first response of the analytical test strip (for example an electrochemical response from electrodes in the first capillary sample-receiving chamber) and determining an analyte in the bodily fluid sample is determined based on the first measured electrochemical response (see steps 920 and 930 of FIG. 9).

In steps 940 and 950 of method 900 also includes, measuring a second response of the analytical test strip (for example, an electrical response from electrodes in the second capillary sample-receiving chamber) and determining a characteristic of the bodily fluid sample based on the second measured response. The measuring and determination steps described above can, if desired, by performed using a suitable associated meter and measurement steps 920 and 930 can be performed in any suitable sequence or in an overlapping manner.

Once apprised of the present disclosure, one skilled in the art will recognize that method 900 can be readily modified to incorporate any of the techniques, benefits and characteristics of analytical test strips according to embodiments of the present invention and described herein.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that devices and methods within the scope of these claims and their equivalents be covered thereby.

What is claimed is:

1. An analytical test strip for the determination of an analyte in a bodily fluid sample, the analytical test strip comprising:
a first capillary sample-receiving chamber;
a second capillary sample-receiving chamber;
a first stop junction disposed between the first capillary sample-receiving chamber and the second capillary receiving chamber and defining a discontinuity boundary of the first capillary sample-receiving chamber; and
a second stop junction disposed between the first capillary sample receiving chamber and the second capillary receiving chamber and defining a discontinuity boundary of the second capillary receiving chamber, and wherein the first stop junction and the second stop junction are disposed such that bodily fluid sample flow between the first capillary sample-receiving chamber and the second capillary sample-receiving chamber during use of the analytical test strip is prevented.

2. The analytical test strip of claim 1 wherein the first capillary sample receiving chamber has at least one sample application opening and the second sample receiving chamber has at least one sample application opening, and
wherein the sample application opening of the first capillary sample-receiving chamber and the sample application opening of the second sample-receiving chamber are juxtaposed such that a single bodily fluid sample can be simultaneously applied thereto.

3. The analytical test strip of claim 2 wherein the first stop junction and the second stop junction extend longitudinally along the first capillary sample-receiving chamber and the second capillary sample-receiving chamber longitudinal length from the sample application opening.

4. The analytical test strip of claim 1 wherein the discontinuity boundary of the first capillary sample-receiving chamber is an increase in a cross-sectional dimension of the first capillary sample-receiving chamber and the discontinuity boundary of the second capillary sample-receiving chamber is an increase in a cross-sectional dimension of the second capillary sample-receiving chamber.

5. The analytical test strip of claim 1 further including:
an electrically insulating substrate layer;
a patterned conductor layer disposed over the electrically-insulating substrate layer, the patterned conductive layer including a plurality of electrodes;
a patterned insulation layer with a first electrode exposure window and a second electrode exposure window an enzymatic reagent layer disposed over at least one of the first electrode exposure window and the second electrode exposure window; and
a patterned spacer layer,
a hydrophilic layer; and
a top layer
wherein at least the electrically-insulating substrate layer, patterned insulation layer, patterned spacer layer, hydrophilic layer and top layer define the first capillary sample-receiving chamber, the second capillary sample-receiving chamber, the first stop junction and the second stop junction.

6. The analytical test strip of claim 5 wherein the analytical test strip further includes:
a hydrophobic layer and,
wherein the discontinuity boundary of the first capillary sample-receiving chamber is defined by an increase in hydrophobicity of the first capillary sample-receiving chamber due to the hydrophobic layer and wherein the discontinuity boundary of the second capillary sample-receiving chamber is defined by an increase in hydrophobicity of the second capillary sample-receiving chamber due to the hydrophobic layer.

7. The analytical test strip of claim 5 wherein the analytical test strip further includes:
a hydrophobic layer and,
wherein the discontinuity boundary of the first capillary sample-receiving chamber is defined by both an increase in hydrophobicity due to the hydrophobic layer and in increase in a dimension of the first capillary sample-receiving chamber, and
wherein the discontinuity boundary of the second capillary sample-receiving chamber is defined by both an increase in hydrophobicity due to the hydrophobic layer and in increase in a dimension of the second capillary sample-receiving chamber

8. The analytical test strip of claim 5 wherein the analytical test strip further includes:
a first hydrophobic layer; and
a second hydrophobic layer, and
wherein the discontinuity boundary of the first capillary sample-receiving chamber is defined by both an increase in hydrophobicity due to the first hydrophobic layer and the second hydrophobic layer and an increase in a dimension of the first capillary sample-receiving chamber, and
wherein the discontinuity boundary of the second capillary sample-receiving chamber is defined by both an increase in hydrophobicity due to the first hydrophobic layer and the second hydrophobic layer and in increase in a dimension of the second capillary sample-receiving chamber.

9. The analytical test strip of claim 1 wherein the analytical test strip is configured as an electrochemical-based analytical test strip.

10. The analytical test strip of claim 1 wherein the bodily fluid sample is a whole blood sample.

11. The analytical test strip of claim 1 wherein the analyte is glucose.

12. The analytical test strip of claim 1 wherein the analyte is glucose and the analytical test strip is configured to determine the analyte in a bodily fluid sample introduced to the first capillary sample-receiving chamber and hematocrit of a bodily fluid sample introduced into the second capillary sample-receiving chamber.

13. A method for determining an analyte in a bodily fluid sample, the method comprising:
applying a bodily fluid sample to an analytical test strip such that the applied bodily fluid sample fills a first capillary sample-receiving chamber and a second capillary sample-receiving chamber of the analytical test strip and is prevented from flowing between the first capillary sample-receiving chamber and the second capillary sample-receiving chamber by at least one stop junction of either of the first capillary sample-receiving chamber and the second capillary sample-receiving chamber;
measuring at least a first response of the analytical test strip; and
determining the analyte based on the first measured electrochemical response.

14. The method of claim 13 further including:
measuring a second response of the analytical test strip that is dependent on bodily fluid sample in the second capillary sample-receiving chamber; and
determining a characteristic of the bodily fluid sample based on the second measured response.

15. The method of claim 13 wherein the bodily fluid sample is whole blood.

16. The method of claim 13 wherein the analyte is glucose.

17. The method of claim 13 wherein the applying step includes applying a single bodily fluid sample to a sample application area of the first capillary sample receiving chamber and a sample application area of the second capillary sample-receiving chamber, and
wherein the sample application opening of the first capillary sample-receiving chamber and the sample application opening of the second sample-receiving chamber are juxtaposed such that the single bodily fluid sample can be simultaneously applied thereto.

18. The method of claim 13 wherein the first stop junction and the second stop junction extend longitudinally along the first capillary sample-receiving chamber and the second capillary sample-receiving chamber longitudinal length from the sample application opening.

19. The method of claim 13 wherein the at least one stop junction forms a discontinuity boundary of the first capillary sample-receiving chamber and the discontinuity boundary is an increase in a cross-sectional dimension of the first capillary sample-receiving chamber.

20. The analytical test strip of claim 19 wherein the at least one stop junction forms a discontinuity boundary of the first capillary sample-receiving chamber and the discontinuity boundary of the first capillary sample-receiving chamber is defined by an increase in hydrophobicity of the first capillary sample-receiving chamber due to the presence of a hydrophobic layer of the analytical test strip.

21. The method of claim 20 wherein the at least one stop junction forms a discontinuity boundary of the first capillary sample-receiving chamber and the discontinuity boundary of the first capillary sample-receiving chamber is defined by both an increase in hydrophobicity of the first capillary sample-receiving chamber due to the presence of a hydrophobic layer of the analytical test strip and in increase in a dimension of the first capillary sample-receiving chamber.

22. The method of claim 20 wherein the at least one stop junction forms a discontinuity boundary of the first capillary sample-receiving chamber and the discontinuity boundary of the first capillary sample-receiving chamber is defined by both an increase in hydrophobicty of the first capillary sample-receiving chamber due to the presence of a first hydrophobic layer and a second hydrophobic layer of the analytical test strip and in increase in a dimension of the first capillary sample-receiving chamber.

23. The method of claim 13 wherein the analytical test strip is configured as an electrochemical-based analytical test strip.
